(19) United States
(12) Patent Application Publication
(10) Pub. No.: US 2005/0287100 A1
(43) Pub. Date: Dec. 29, 2005

(54) Cosmetic composition comprising a semi-crystalline polymer and a dispersion of polymer in fatty phase

(76) Inventor: Caroline Lebre, Thiais (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/147,318

(22) Filed: Jun. 8, 2005

Related U.S. Application Data
(60) Provisional application No. 60/580,364, filed on Jun. 18, 2004.

(30) Foreign Application Priority Data
Jun. 8, 2004 (FR) 04 06169

(51) Int. Cl. A61K 7/06; A61K 7/11

(52) U.S. Cl. 424/70.16

(57) ABSTRACT

The present disclosure relates to a cosmetic composition comprising at least one fatty phase, a dispersion of particles of at least one polymer dispersed in the at least one fatty phase, and at least one semi-crystalline polymer of organic structure whose melting point is greater than or equal to 30° C., wherein the at least one fatty phase contains less than 50% by weight of at least one volatile oil.

This application claims benefit of U.S. Provisional Application No. 60/580,364, filed Jun. 18, 2004, the contents of which are incorporated herein by reference. This application also claims benefit of priority under 35 U.S.C. § 119 to French Patent Application No. 04 06169, filed Jun. 8, 2004, the contents of which are also incorporated by reference.

The present disclosure relates to a composition for use in cosmetics, comprising polymer particles dispersed in a fatty phase. The composition may also comprise a semi-crystalline polymer.

In one aspect of the present disclosure, the disclosure relates to a care or makeup composition for keratin materials, such as the skin of either the human face or body, including the scalp, the integuments, for instance the eyelashes, the eyebrows, the nails and the hair, and also the lips and the eyelids.

Cosmetic compositions are usually based on gelled or non-gelled oils and on pasty compounds. Oils that ensure a desired gloss effect generally have a high refractive index and high viscosity.

However, since the predominant constituents of these formulations are liquid at the temperature of the lips, they have a tendency to escape beyond their contour via the fine lines, giving an unattractive effect or discomfort, in particular to the eyes, which users do not appreciate. Moreover, the staying power of these formulations over time is often disappointing.

Similarly, the oils predominantly used in compositions of this type are usually viscous (in order to achieve the desired gloss result) and can thereby also be responsible for the undesirable tacky nature of the deposit.

The inventors have discovered, surprisingly, that by combining a dispersion of polymer particles in a fatty phase with a semi-crystalline polymer, formulations with improved staying power, e.g., having a low level of migration and of tack may be obtained.

The term “deposit with good staying power” means a makeup whose color and/or gloss persists throughout the day, and which withstands ordeals, such as mealtimes in the case of a lipstick.

U.S. Pat. Nos. 5,736,125 and 5,156,911, and also patent application WO 01/19333, illustrate certain types of semi-crystalline polymer that may be included in the composition of the formulations of the disclosure. However, these documents do not describe cosmetic compositions, and do not suggest that the addition of a dispersion of polymer particles to this type of polymer makes it possible to obtain a deposit on keratin materials that can have improved staying power and that can be glossy, tack-free and that generally does not migrate.

U.S. Pat. No. 6,464,969 describes mascara compositions containing a polymer in dispersion and a heat-transition agent that may be polycaprolactone. However, these compositions contain a large proportion of volatile oil.

One embodiment of the present disclosure is a cosmetic composition comprising at least one fatty phase, a dispersion of particles of at least one polymer dispersed in the at least one fatty phase, and at least one semi-crystalline polymer of organic structure whose melting point is greater than or equal to 30° C., the at least one fatty phase being free of volatile oil or containing less than 50% by weight of at least one volatile oil relative to the weight of the at least one fatty phase.

The composition of the disclosure may take any art-recognized form, for example, a paste, a solid or a cream. It may be an oil-in-water or water-in-oil emulsion, or a solid or soft anhydrous gel. In one embodiment, the composition may be in anhydrous form, such as in the form of an anhydrous gel, and, in one embodiment, the composition may be cast in a stick or a dish.

As used herein, the term “fatty phase” means any non-aqueous medium.

In an embodiment of the present disclosure, the at least one fatty phase may be at least partially liquid at room temperature (25° C.) and atmospheric pressure (760 mmHg) and may be composed of one or more fatty substances that are liquid at room temperature, also known as oils.

Semi-Crystalline Polymer

As used herein, the term “polymer” means a compound containing at least two repeating units, such as at least three repeating units, or up to and including more than at least ten repeating units.

In the present disclosure, the term “semi-crystalline polymer” means at least one polymer comprising a crystallizable portion and an amorphous portion in the skeleton and having a first-order reversible change of phase temperature, such as melting (solid-liquid transition). The crystallizable portion may be either a side chain (or pendent chain) or a block in the skeleton.

When the crystallizable portion of the at least one semi-crystalline polymer is a block of the polymer skeleton, this crystallizable block may have a different chemical nature from that of the amorphous blocks; in this case, the semi-crystalline polymer may be a block copolymer, for example of the diblock, triblock or multiblock type. When the crystallizable portion is a chain that is pendent on the skeleton, the semi-crystalline polymer may be a homopolymer or a copolymer.

The terms “organic compound” and “having an organic structure” mean compounds containing carbon atoms and hydrogen atoms and optionally hetero atoms such as S, O, N or P, alone or in combination.

In one embodiment of the present disclosure, the melting point of the at least one semi-crystalline polymer may be less than 150° C.

In one embodiment of the present disclosure, the melting point of the at least one semi-crystalline polymer may range from 30° C. to 100° C. In yet another embodiment, the melting point of the at least one semi-crystalline polymer may range from 30° C. to 60° C.

The at least one semi-crystalline polymer which may be used according to the present disclosure may be solid at room temperature (25° C.) and atmospheric pressure (760 mmHg), with a melting point of greater than or equal to 30° C. The melting point values correspond to the melting point measured using a differential scanning calorimeter (DSC), such as the calorimeter sold under the name DSC 30 by the company Mettler, with a temperature rise of 5° C. or 10° C. per minute (the melting point under consideration is the point corresponding to the temperature of the most endothermic peak of the thermogram).

The at least one semi-crystalline polymers which may be used according to the disclosure may have a melting point that is higher than the temperature of the keratinous support intended to receive the composition, such as the skin or the lips.

In one embodiment of the present disclosure, the at least one semi-crystalline polymers may be capable, alone or as a mixture, of structuring the composition without the addition of a particular surfactant and/or a filler and/or a wax.

According to the present disclosure, the at least one semi-crystalline polymer may be soluble in the at least one fatty phase, such as, for example, at least 1% by weight, at a temperature that is higher than their melting point. Besides the crystallizable chains or blocks, the blocks of the polymers may be amorphous.

As used herein, the expression “crystallizable chain or block” means a chain or block which, if it were obtained alone, would change from the amorphous state to the crystalline state reversibly, depending on whether one is above or below the melting point. A “chain” is a group of atoms, which are pendent or lateral relative to the polymer skeleton. A “block” is a group of atoms belonging to the skeleton, this group constituting one of the repeating units of the polymer.

In one embodiment of the present disclosure, the polymer skeleton of the at least one semi-crystalline polymers may be soluble in the at least one fatty phase.

In one embodiment of the present disclosure, the crystallizable blocks or chains of the at least one semi-crystalline polymer may be present in an amount comprising at least 30% of the total weight of each polymer, such as, for example, at least 40% of the total weight of each polymer. The at least one semi-crystalline polymer containing crystallizable side chains may be homopolymers or copolymers. The at least one semi-crystalline polymer of the present disclosure containing crystallizable blocks may be a block or a multi-block copolymer. It may be obtained by polymerizing a monomer containing reactive (or ethylenic) double bonds or by polycondensation. When the polymer of the disclosure is a polymer containing crystallizable side chains, these side chains may be in random or statistical form.

In one embodiment, the at least one semi-crystalline polymer of the present disclosure may be of synthetic origin. According to another embodiment of the disclosure, the at least one semi-crystalline polymers of the disclosure may not comprise a polysaccharide skeleton.

The at least one semi-crystalline polymer that may be used in the disclosure may be chosen from, one or more of:
block copolymers of polyolefins of controlled crystallization, whose monomers are described in European Patent Publication No. 0 951 897;
polycondensates, such as polycondensates of the aliphatic or aromatic polyester type or of aliphatic/aromatic polyester type;
homopolymers or copolymers bearing at least one crystallizable side chain and homopolymers or copolymers bearing at least one crystallizable block in the skeleton, for instance those described in document U.S. Pat. No. 5,156,911;
homopolymers or copolymers bearing at least one crystallizable side chain, in particular bearing one or more fluoro groups, such as those described in WO 01/19333.

In the last two cases, the crystallizable side chains or block may be hydrophobic.

A) At Least One Semi-Crystalline Polymer Containing at Least One Crystallizable Side Chain

According to one embodiment, the at least one semi-crystalline polymer containing at least one crystallizable side chain can be defined as in U.S. Pat. No. 5,156,911 and WO 01/19333. These polymers are homopolymers or copolymers comprising from 50% to 100% by weight of units resulting from the polymerization of one or more monomers bearing a crystallizable hydrophobic side chain.

These homopolymers or copolymers can be of any nature, provided that they meet the conditions mentioned hereinbelow, for example being soluble or dispersible in the at least one fatty phase by heating above their melting point (mp).

Such homopolymers or copolymers can result from:
the polymerization, especially the free-radical polymerization, of one or more monomers containing reactive or ethylenic double bond(s) with respect to a polymerization, namely a vinyl, (meth)acrylic or allylic group,
the polycondensation of one or more monomers bearing co-reactive groups (carboxylic acid, sulfonic acid, alcohol, amine or isocyanate), such as, for example, polyesters, polyurethanes, polyethers, polyureas or polyamides.

The crystallizable units (chains or blocks) of the at least one semi-crystalline polymer according to the present disclosure may be derived from monomers containing crystallizable blocks or chains, such as those used for manufacturing semi-crystalline polymers. These polymers may be chosen from homopolymers and copolymers resulting from the polymerization of at least one monomer containing crystallizable chains that may be represented by formula X: 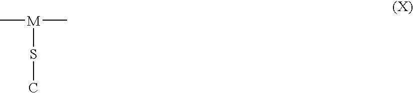
wherein:
M is an atom of the polymer skeleton;
S is a spacer; and
C is a crystallizable group.

The crystallizable chains “—S—C” may be aliphatic or aromatic, and optionally fluorinated or perfluorinated. In one embodiment, “S” may be a group (CH₂)ₙ or (CH₂CH₂O)ₙ or (CH₂O), which may be linear or branched or cyclic, with n being an integer ranging from 0 to 22. In another embodiment, “S” may be a linear group. In yet another embodiment of the present disclosure, “S” and “C” are different.

When the crystallizable chains are hydrocarbon-based aliphatic chains, they may comprise hydrocarbon-based alkyl chains containing at least 11 carbon atoms and not more than 40 carbon atoms, or, for example, not more than 24 carbon atoms. In one embodiment, the crystallizable chains may be aliphatic chains or alkyl chains containing at least 12 carbon atoms, such as C₁₄-C₂₄ or C₁₆-C₂₂ alkyl chains. When they are fluoroalkyl or perfluoroalkyl chains, they may contain at least 11 carbon atoms, at least 6 of which carbon atoms may be fluorinated.

As examples of semi-crystalline homopolymers or copolymers containing crystallizable chains, non-limiting mention may be made of those resulting from the polymerization of one or more of the following monomers: (meth)acrylates of saturated alkyls with the alkyl group being C₁₄-C₂₄, perfluoroalkyl (meth)acrylates with a C₁₁-C₁₅ perfluoroalkyl group, N-alkyl(meth)acrylamides with the alkyl group being C₁₄ to C₂₄ optionally comprising a fluorine atom, vinyl esters containing alkyl or perfluoroalkyl chains with the alkyl group being C₁₄ to C₂₄ (with at least 6 fluorine atoms per perfluoroalkyl chain), vinyl ethers containing alkyl or perfluoroalkyl chains with the alkyl group being C₁₄ to C₂₄ and at least 6 fluorine atoms per perfluoroalkyl chain, C₁₄ to C₂₄ α-olefins such as, for example, octadecene, para-alkylstyrenes with an alkyl group containing from 12 to 24 carbon atoms, and mixtures thereof.

When the polymers result from a polycondensation, the hydrocarbon-based and/or fluorinated crystallizable chains as defined above may be borne by a monomer that may be a diacid, a diol, a diamine or a diisocyanate. When the polymers that are the subject of the disclosure are copolymers, they may additionally comprise from 0 to 50% of groups Y or Z resulting from the copolymerization:

Y may be a polar or non-polar monomer or a mixture of the two:

When Y is a polar monomer, it may be either a monomer bearing polyoxyalkylenated groups (such as oxyethylenated and/or oxypropylenated groups), a hydroxyalkyl (meth)acrylate, for instance hydroxyethyl acrylate, (meth)acrylamide, an N-alkyl(meth)acrylamide, an N,N-dialkyl(meth)acrylamide such as, for example, N,N-diisopropylacrylamide or N-vinylpyrrolidone (NVP), N-vinylcaprolactam, a monomer bearing at least one carboxylic acid group, for instance (meth)acrylic acid, crotonic acid, itaconic acid, maleic acid or fumaric acid, or bearing a carboxylic acid anhydride group, for instance maleic anhydride, and mixtures thereof.

When Y is a non-polar monomer, it may be an ester of the linear, branched or cyclic alkyl (meth)acrylate type, a vinyl ester, an alkyl vinyl ether, an α-olefin, styrene or styrene substituted with a C₁ to C₁₀ alkyl group, for instance α-methylstyrene, or a macromonomer of the polyorganosiloxane type containing vinyl unsaturation and mixtures thereof.

As used herein, the term “alkyl” means a saturated group, such as a C₈ to C₂₄, except where otherwise mentioned.

Z may be a polar monomer or a mixture of polar monomers. In this case, Z has the same definition as the “polar Y” defined above.

In one embodiment, the at least one semi-crystalline polymer containing at least one crystallizable side chain may be alkyl (meth)acrylate or alkyl(meth)acrylamide homopolymers with an alkyl group as defined above, such as C₁₄-C₂₄, copolymers of these monomers with a hydrophilic monomer which may have a different nature from (meth)acrylic acid, for instance N-vinylpyrrolidone or hydroxyethyl (meth)acrylate.

According to one embodiment of the present disclosure, the at least one semi-crystalline polymer containing at least one crystallizable side chain may have a weight-average molar mass (Mp) ranging from 5000 to 1,000,000, such as from 10,000 to 800,000. In other embodiments of the present disclosure, the at least one semi-crystalline polymer containing at least one crystallizable side chain may have a weight-average molar mass (Mp) ranging from 15,000 to 500,000, or from 100,000 to 200,000.

Among examples of the at least one semi-crystalline polymer that may be used in the composition according to the disclosure, non-limiting mention may be made of the Intelimer® products from the company Landec described in the brochure “Intelimer® polymers”, Landec IP22 (Rev. 4-97). These polymers are in solid form at room temperature (25° C.). They bear crystallizable side chains and have the above formula X.

For example, the at least one semi-crystalline polymer may be the Intelimer® product IPA 13-1 from the company Landec may be used, which is a polystearyl acrylate with a molecular weight of about 145,000 and a melting point of 49° C.

Other semi-crystalline polymers that may be used according the present disclosure include those described in Examples 3, 4, 5, 7 and 9 of U.S. Pat. No. 5,156,911, containing a —COOH group, resulting from the copolymerization of acrylic acid and of a C₅ to C₁₆ alkyl (meth)acrylate with a melting point ranging from 20° C. to 35° C., as well as those from the copolymerization of:
acrylic acid, of hexadecyl acrylate and of isodecyl acrylate in a 1/16/3 ratio;
acrylic acid and of pentadecyl acrylate in a 1/19 ratio;
acrylic acid, of hexadecyl acrylate and of ethyl acrylate in a 2.5/76.5/20 ratio;
acrylic acid, of hexadecyl acrylate and of methyl acrylate in a 5/85/10 ratio; and
acrylic acid and of polyoctadecyl (meth)acrylate in a 2.5/97.5 ratio.

Another example of the at least one semi-crystalline polymer that may be used includes the polymer Structure “O” from National Starch, such as the product described in U.S. Pat. No. 5,736,125 with a melting point of 44° C.

The at least one semi-crystalline polymer may be a semi-crystalline polymer with crystallizable pendent chains comprising fluoro groups, such as those described in Examples 1, 4, 6, 7 and 8 of WO 01/19333.

In another embodiment of the present disclosure, the at least one semi-crystalline polymer may be chosen from those obtained by copolymerization of stearyl acrylate and of acrylic acid or of NVP, as described in U.S. Pat. No. 5,519,063 and European Patent Publication EP550 745.

Non-limiting mention may also be made of semi-crystalline polymers obtained by copolymerization of behenyl acrylate and of acrylic acid or of NVP, as described in U.S. Pat. No. 5,519,063 and European Patent Publication EP 550 745, as well as those for which the method of preparation is described in Examples 3 and 4 below.

B) Polymers Bearing at Least One Crystallizable Block in the Skeleton

When the at least one semi-crystalline polymer is a polymer that bears at least one crystalliable block, it may be soluble or dispersible in the at least one fatty phase by heating above their melting point (mp). These polymers may be block copolymers including at least two blocks of different chemical nature, one of which may be crystallizable.

The at least one semi-crystalline polymer bearing at least one crystallizable block in the skeleton may be chosen from block copolymers of olefin or of cycloolefin containing a crystallizable chain, for instance those derived from the block polymerization of:
cyclobutene, cyclohexene, cyclooctene, norbornene (i.e. bicyclo(2,2,1)-2-heptene), 5-methylnorbornene, 5-ethylnorbornene, 5,6-dimethylnorbornene, 5,5,6-trimethylnorbornene, 5-ethylidenenorbornene, 5-phenylnorbornene, 5-benzylnorbornene, 5-vinyinorbornene, 1,4,5,8-dimethano-1,2,3,4,4a,5,8α-octahydronaphthalene, dicyclopentadiene, or mixtures thereof;
with ethylene, propylene, 1-butene, 3-methyl-1-butene, 1-hexene, 4-methyl-1-pentene, 1-octene, 1-decene or 1-eicosene, or mixtures thereof;
copoly(ethylene/norbornene) blocks; and
(ethylene/propylene/ethylidene-norbornene) block terpolymers.
   The at least one semi-crystalline polymer may result from the block copolymerization of at least two C₂-C₁₆, or C₂-C₁₂ α-olefins such as those mentioned above, as well as bipolymers of ethylene and of 1-octene.

The at least one semi-crystalline polymer bearing at least one crystallizable block in the skeleton may be chosen from copolymers containing at least one crystallizable block, the rest of the copolymer being amorphous (at room temperature). According to one embodiment, these copolymers may also contain two crystallizable blocks of different chemical nature.

The at least one semi-crystalline polymer may be chosen from copolymers including those that simultaneously contain at room temperature a crystallizable block and an amorphous block that are both hydrophobic and lipophilic, sequentially distributed; mention may also be made, for example, of polymers containing one of the crystallizable blocks and one of the amorphous blocks chosen from the blocks described below:
Blocks that are crystallizable by nature, of polyester type, for instance poly(alkylene terephthalate), or of polyolefin type, for instance polyethylenes or polypropylenes.
Amorphous and lipophilic blocks, for instance: amorphous polyolefins or copoly(olefin)s such as poly(isobutylene), hydrogenated polybutadiene or hydrogenated poly(isoprene).

As examples of such copolymers containing a crystallizable block and an amorphous block, mention may be made of:
a) poly(ε-caprolactone)-b-poly(butadiene) block copolymers, preferably used hydrogenated, such as those described in the article “Melting behavior of poly(-caprolactone)-block-polybutadiene copolymers” from S. Nojima, Macromolecules, 32, 3727-3734 (1999),
b) the hydrogenated block or multiblock poly(butylene terephthalate)-b-poly(isoprene) block copolymers cited in the article “Study of morphological and mechanical properties of PP/PBT” by B. Boutevin et al., Polymer Bulletin, 34, 117-123 (1995),
c) the poly(ethylene)-b-copoly(ethylene/propylene) block copolymers cited in the articles “Morphology of semi-crystalline block copolymers of ethylene-(ethylene-alt-propylene)” by P. Rangarajan et al., Macromolecules, 26, 4640-4645 (1993) and “Polymer aggregates with crystalline cores: the system poly(ethylene)-poly(ethylene-propylene)” by P. Richter et al., Macromolecules, 30, 1053-1068 (1997),
d) the poly(ethylene)-b-poly(ethylethylene) block copolymers cited in the general article “Crystallization in block copolymers” by I. W. Hamley, Advances in Polymer Science, Vol. 148, 113-137 (1999).
   C) Polycondensates of Aliphatic or Aromatic or Aliphatic/Aromatic Polyester Type

The polyester polycondensates that may be used as the at least one semi-crystalline polymer may be chosen from aliphatic polyesters. Their molar mass may range from 200 to 10,000 g/mol. In one embodiment, their molar mass may range from 300 to 5000 g/mol. In another embodiment, their molar mass may range from 500 to 2000 g/mol.

The polyester polycondensates may be chosen from polycaprolactones. In one embodiment, the polycaprolactones may be chosen from ε-caprolactone homopolymers. The homopolymerization may be initiated with, for example, a diol, such as a diol containing from 2 to 10 atoms, such as diethylene glycol, 1,4-butanediol or neopentyl glycol.

Polycaprolactones that may be used, for example, include those sold under the name CAPA® 240 (melting point of 68° C. and molecular weight of 4000), CAPA® 223 (melting point of 48° C. and molecular weight of 2000), CAPA® 222 (melting point of 48° C. and molecular weight of 2000), CAPA® 217 (melting point of 44° C. and molecular weight of 1250), CAPA® 2125 (melting point of 45° C. and molecular weight of 1250), CAPA® 212 (melting point of 45° C. and molecular weight of 1000), CAPA® 210 (melting point of 38° C. and molecular weight of 1000), CAPA® 205 (melting point of 39° C. and molecular weight of 830) by the company Solvay, or PCL-300 and PCL-700 by the company Union Carbide.

The CAPA® 2125 whose melting point is between 35 and 45° C. and whose molecular weight is equal to 1250 may be used in one embodiment of the present disclosure.

The at least one semi-crystalline polymer in the composition of the disclosure may or may not be partially crosslinked, provided that the degree of crosslinking does not interfere with its dissolution or dispersion in the at least one fatty phase by heating above its melting point. The at least one semi-crystalline polymer may be chemically crosslinked, by reaction with a multifunctional monomer during the polymerization. It may also be physically crosslinked, for example by either 1) the establishment of bonds of hydrogen or dipolar type between groups borne by the polymer, such as, for example, the dipolar interactions between carboxylate ionomers, these interactions being of small amount and borne by the polymer skeleton; or 2) a phase separation between the crystallizable blocks and the amorphous blocks borne by the polymer.

In one embodiment, the at least one semi-crystalline polymer in the composition according to the disclosure is non-crosslinked.

In one embodiment of the present disclosure, the at least one semi-crystalline polymers may be present in an amount ranging from 0.1% to 80% of the total weight of the composition. In another embodiment of the present disclosure, the at least one semi-crystalline polymer may be present in an amount ranging from 0.5% to 40%, or from 3% to 30% of the total weight of the composition. In another embodiment of the present disclosure, the at least one semi-crystalline polymer is present in an amount ranging from 5% to 25% of the total weight of the composition.

In one embodiment, the polymer, when dispersed in sufficient amount in the at least one fatty phase, exhibits a mean gloss at 20° of a deposit of the composition, once spread onto a support, of greater than or equal to 30 out of 100.

Another embodiment of the present disclosure relates to a cosmetic composition containing particles of at least one polymer dispersed in at least one liquid fatty phase, the phase containing at least one non-volatile hydrocarbon-based oil that is present in an amount comprising at least 5% by weight of the composition. According to one embodiment, the polymer and the non-volatile hydrocarbon-based oil are present in a sufficient amount so that the composition exhibits a mean gloss at 20° of a deposit of the said composition, once spread onto a support, of greater than or equal to 30 out of 100.

The term “mean gloss” means the gloss as may be conventionally measured using a glossmeter by the following method.

A coat of between 50 μm and 150 μm in thickness of the composition is spread using an automatic spreader onto a Leneta brand contrast card of reference Form 1A Penopac. The coating covers at least the white background of the card. When the composition is solid it is melted, if necessary, on the card after it has been spread and has covered the white background.

The deposit is left to dry for 24 hours at a temperature of 30° C., and the gloss at 200 is then measured on the white background using a Byk Gardner brand glossmeter of reference microTri-Gloss.

This measurement (between 0 and 100) is repeated at least three times, and the mean gloss is the mean of the at least three measurements taken.

The mean gloss of the composition measured at 200 may be greater than or equal to 30, or greater than or equal to 35 out of 100. In one embodiment, the mean gloss may be greater than or equal to 40, greater than or equal to 45, greater than or equal to 50, greater than or equal to 55, or greater than or equal to 60 out of 100.

In one embodiment, the mean gloss of the composition, once spread onto a support, measured at 60° is greater than or equal to 50, greater than or equal to 60, greater than or equal to 65, greater than or equal to 70, greater than or equal to 75, greater than or equal to 80, greater than or equal to 85, or greater than or equal to 90 out of 100.

The mean gloss at 60° is measured as described above, by taking the measurement at 60° rather than at 20°.

According to one embodiment, the mean gloss of the composition measured at 20° may be greater than or equal to 35, 40, 45 or 50 out of 100, and/or the mean gloss of the composition measured at 60° may be greater than or equal to 65, 70 or 75 out of 100. In this embodiment, the composition may constitute a liquid lipstick.

The disclosure also relates to the cosmetic use of the composition defined above for improving the gloss of a deposit on the skin and/or the lips and/or the integuments, for example, a makeup.

As used herein, the term “liquid fatty phase” means any non-aqueous medium.

In one embodiment, the at least one fatty phase may be at least partially liquid at room temperature (25° C.) and atmospheric pressure (760 mmHg) and may be composed of one or more fatty substances that are liquid at room temperature, also known as oils.

According to one embodiment of the composition according to the disclosure, the dispersion of polymer particles in at least one apolar or sparingly polar oil may be such that when they are in a sufficient amount in the composition, this composition may be capable of forming a deposit that has a wear index of greater than or equal to 30%, greater than or equal to 40%, greater than or equal to 45%, or greater than or equal to 50%.

The wear index of the deposit obtained with the composition according to the disclosure is determined according to the measuring protocol described below.

A support (40 mm×70 mm rectangle) is prepared including an acrylic coating (hypoallergenic acrylic adhesive on polyethylene film sold under the name Blenderme ref. FH5000-55113 by the company 3M Sante) bonded onto a layer of polyethylene foam that is adhesive on the side opposite the one to which the adhesive plaster is fixed (foam layer sold under the name RE 40X70EP3 from the company Joint Technique Lyonnais Ind.).

The color L*₀a*₀b*₀ of the support, on the acrylic coating side, is measured using a Minolta CR300 calorimeter.

The support thus prepared is preheated on a hotplate maintained at a temperature of 40° C. so that the surface of the support is maintained at a temperature of 33° C.±1° C.

While leaving the support on the hotplate, the composition is applied to the entire non-adhesive surface of the support (i.e. to the surface of the acrylic coating), spreading it out with a brush to obtain a deposit of the composition of about 15 μm, and this deposit is then left to dry for 10 minutes.

After drying, the color L*a*b* of the film thus obtained is measured.

The color difference ΔE1 between the color of the film relative to the color of the naked support is then determined via the following relationship:
ΔE1=\sqrt{(L*−L₀*)²+(a*−a₀*)²+(b*−b₀*)²}

The support is then bonded via its adhesive face (adhesive face of the foam layer) to an anvil 20 mm in diameter and equipped with a screw pitch. A sample of the support/deposit assembly is then cut out using a sample punch 18 mm in diameter. The anvil is then screwed onto a press (Statif Manuel Imada SV-2 from the company Someco) equipped with a tensile testing machine (Imada DPS-20 from the company Someco).

A strip 33 mm wide and 29.7 cm long is drawn on a sheet of white photocopier paper with a basis weight of 80 g/m², a first line is marked 2 cm from the edge of the sheet, and a second line is then marked 5 cm from the edge of the sheet, the first and second lines thus delimiting a box on the strip; next, a first mark and a second mark located in the strip at reference points 8 cm and 16 cm, respectively, from the second mark, are applied. 20 μl of water are placed on the first mark and 10 μl of refined sunflower oil (sold by the company Lesieur) are placed on the second mark.

The white paper is placed on the base of the press and the sample placed on the box of the strip of paper is then pressed at a pressure of about 300 g/cm² exerted for 30 seconds. The press is then opened and the sample is again placed just after the second mark (i.e. next to the box), a pressure of about 300 g/cm² is again exerted, and the paper is displaced, in a rectilinear manner as soon as the contact is made, at a speed of 1 cm/s over the entire length of the strip such that the sample passes through the water and oil deposits.

After removing the sample, some of the deposit has transferred onto the paper. The color L*′, a*′, b*′ of the deposit remaining on the sample is then measured.

The color difference ΔE2 between the color of the deposit remaining on the sample relative to the color of the naked support is then determined via the following relationship
ΔE2=\sqrt{(L*′−L₀*)²+(a*′−a₀*)²+(b*′−b₀*)²}

The wear index of the composition, expressed as a percentage, is equal to the ratio:
100×ΔE2/ΔE1

The measurement is performed on 6 supports in succession and the wear index corresponds to the mean of the six measurements obtained with the six supports.

At Least One Polymer in Dispersion

According to the disclosure, the at least one polymer may be a solid that is insoluble in the at least one liquid fatty phase of the composition at room temperature, for example at approximately 25° C. The at least one polymer may also be insoluble in the at least one liquid fatty phase at its softening point, unlike a wax, which is soluble in the at least one fatty phase at a temperature above its melting point. In this sense, the at least one polymer is not a wax.

The at least one polymer in dispersion may also allow the formation of a deposit that is continuous and homogeneous and/or is characterized by the overlapping of the polymer chains.

The composition according to the disclosure may comprise at least one stable dispersion of essentially spherical polymer particles of one or more polymers, in the at least one liquid fatty phase.

These dispersions may be in the form of polymer nanoparticles in stable dispersion in the liquid organic phase. The nanoparticles may have a mean size ranging from 5 to 800 nm, such as from 50 to 500 nm. In other embodiments, the polymer particles may range up to 1 μm in size.

In one embodiment, the polymer particles in dispersion may be insoluble in water-soluble alcohols, for instance ethanol.

The at least one polymer in dispersion that may be used in the composition of the disclosure may have a molecular weight ranging from 2000 to 10,000,000 g/mol and a Tg ranging from −100° C. to 300° C., such as, for example, from −50° C. to 100° C., or from −10° C. to 50° C.

The at least one polymer may be film-forming. It is possible to use film-forming polymers having a low Tg, of less than or equal to skin temperature, or less than or equal to 40° C.

The term “film-forming polymer” means a polymer that is capable, by itself or in the presence of an auxiliary film-forming agent, of forming a continuous film that adheres to a support, such as keratin materials, and which may be a cohesive film, or a film whose cohesion and mechanical properties are such that the film can be isolated from the support.

In another embodiment, a non-film-forming polymer may be used. The term “non-film-forming polymer” means a polymer that is incapable by itself of forming an isolable film.

Among the film-forming polymers that may be mentioned are acrylic or vinyl free-radical homopolymers or copolymers, such as those having a Tg of less than or equal to 40° C., including those having a Tg ranging from −10° C. to 30° C., used alone or as a mixture.

Among the non-film-forming polymers that may be mentioned are optionally crosslinked vinyl or acrylic free-radical homopolymers or copolymers that may have a Tg of greater than 40° C., including those having a Tg ranging from 45° C. to 150° C., used alone or as a mixture.

The term “free-radical polymer” means a polymer obtained by polymerization of unsaturated and especially ethylenic monomers, each monomer being capable of homopolymerizing (unlike polycondensates). The free-radical polymers may be vinyl polymers or copolymers, such as acrylic polymers.

The acrylic polymers may resuit from the polymerization of ethylenically unsaturated monomers containing at least one acid group and/or esters of these acid monomers and/or amides of these acids.

Among meth(acrylic)/(meth)acrylate copolymers that may be used in the present disclosure, non-limiting mention may be made of acrylic/acrylate copolymers having a mass ratio of the acrylic units to acrylate units ranging from 0.1% and 40%, from 2% to 30%, and from 5% to 20%.

Monomers bearing an acid group that may be used include α,β-ethylenic unsaturated carboxylic acids such as acrylic acid, (meth)acrylic acid, crotonic acid, maleic acid or itaconic acid. In one embodiment, (meth)acrylic acid and crotonic acid may be used. In yet another embodiment, (meth)acrylic acid may be used.

In one embodiment, the acid monomer esters may be chosen from (meth)acrylic acid esters (also known as (meth)acrylates), for instance alkyl (meth)acrylates, such as C₁-C₂₀ and C₁-C₈ alkyl, aryl (meth)acrylates, including a C₆-C₁₀ aryl, and hydroxyalkyl (meth)acrylates, such as a C₂-C₆ hydroxyalkyl. Alkyl (meth)acrylates that may be mentioned include methyl, ethyl, butyl, isobutyl, 2-ethylhexyl and lauryl (meth)acrylate. Hydroxyalkyl (meth)acrylates that may be mentioned include hydroxyethyl (meth)acrylate and 2-hydroxypropyl (meth)acrylate. Aryl (meth)acrylates that may be mentioned include benzyl or phenyl acrylate.

The (meth)acrylic acid esters that may be used are the alkyl (meth)acrylates.

Free-radical polymers that may be used include copolymers of (meth)acrylic acid and of alkyl (meth)acrylate, such as those of a C₁-C₄ alkyl. Methyl acrylates optionally copolymerized with acrylic acid may also be used.

Amides of the acid monomers that may be mentioned include (meth)acrylamides, such as N-alkyl(meth)acrylamides, for example of a C₂-C₁₂ alkyl, such as N-ethylacrylamide, N-t-butylacrylamide and N-octylacrylamide; N-di(C₁-C₄)alkyl(meth)acrylamides.

The acrylic polymers may also result from the polymerization of ethylenically unsaturated monomers containing at least one amine group, in free form or in partially or totally neutralized form, or alternatively in partially or totally quaternized form. Such monomers may be, for example, dimethylaminoethyl (meth)acrylate, dimethylaminoethyl(meth)acrylamide, vinylamine, vinylpyridine or diallyldimethylammonium chloride.

The vinyl polymers may also result from the homopolymerization or copolymerization of at least one monomer chosen from vinyl esters and styrene monomers. In one embodiment, these monomers may be polymerized with acid monomers and/or esters thereof and/or amides thereof, such as those mentioned previously. Examples of vinyl esters that may be mentioned include vinyl acetate, vinyl propionate, vinyl neodecanoate, vinyl pivalate, vinyl benzoate and vinyl t-butylbenzoate. Styrene monomers that may be mentioned include styrene and α-methylstyrene.

The list of monomers given is not limiting, and it is possible to use any monomer known to those skilled in the art included in the categories of acrylic and vinyl monomers (including monomers modified with a silicone chain).

As other vinyl monomers that may be used, mention may also be made of:
N-vinylpyrrolidone, N-vinylcaprolactam, vinyl-N-(C₁-C₈)alkylpyrroles, vinyloxazoles, vinylthiazoles, vinylpyrimidines and vinylimidazoles, and
olefins such as ethylene, propylene, butylene, isoprene or butadiene.

The vinyl polymer may be crosslinked with one or more difunctional monomers that may comprise at least two ethylenic unsaturations, such as ethylene glycol di(meth)acrylate or diallyl phthalate.

The at least one polymer in dispersion of the disclosure may be chosen from one or more of the following polymers or copolymers: polyurethanes, polyurethane-acrylics, polyureas, polyurea-polyurethanes, polyester-polyurethanes, polyether-polyurethanes, polyesters, polyesteramides, alkyds; acrylic and/or vinyl polymers or copolymers; acrylic-silicone copolymers; polyacrylamides; silicone polymers, for instance silicone polyurethanes or silicone acrylics, and fluoro polymers.

The at least one polymer in dispersion in the at least one liquid fatty phase may be present in an amount ranging from 5% to 40% of the weight of solids in the composition. In one embodiment, the at least one polymer in dispersion in the at least one liquid fatty phase may be present in an amount ranging from 5% to 35%, such as from 8% to 30%, of the weight of solids in the composition.

According to one embodiment, the polymer particles in dispersion may be surface-stabilized with at least one stabilizer that is solid at room temperature. In this case, the amount of solids in the dispersion represents the total amount of polymer +stabilizer, given that the amount of polymer cannot be less than 5%.

In one embodiment, a dispersion of particles of at least one film-forming polymer is used.

At Least One Stabilizer

The polymer particles in organic medium may be surface-stabilized, as the polymerization proceeds, by means of at least one stabilizer that may be a block polymer, a grafted polymer and/or a random polymer. The stabilization may take place by any known means, and may be by direct addition of the block polymer, grafted polymer and/or random polymer during the polymerization.

The at least one stabilizer may also be present in the mixture before polymerization. However, it is also possible to add it continuously, such as when the monomers are also added continuously.

The at least one stabilizer may be used in an amount ranging from 2 to 30% by weight, such as, for example, from 5-20% by weight relative to the initial monomer mixture.

When a grafted polymer and/or a block polymer is used as the at least one stabilizer, the synthesis solvent may be chosen such that at least some of the grafts or blocks of the polymer-stabilizer are soluble in the solvent, the rest of the grafts or blocks being insoluble therein. The polymer-stabilizer used during the polymerization should be soluble, or dispersible, in the synthesis solvent. Furthermore, at least one stabilizer whose insoluble blocks or grafts have a certain affinity for the polymer formed during the polymerization may be chosen.

Among the grafted polymers that may be mentioned are silicone polymers grafted with a hydrocarbon-based chain; hydrocarbon-based polymers grafted with a silicone chain.

Grafted copolymers having, for example, an insoluble skeleton of polyacrylic type with soluble grafts of poly(12-hydroxystearic acid) type are also suitable for use.

Thus, grafted-block or block copolymers comprising at least one block of the polyorganosiloxane type and at least one block of a free-radical polymer, for instance grafted copolymers of the acrylic/silicone type, may thus be used. In one embodiment, these polymers may be used when the synthesis medium and then the organic phase of the first composition contains a silicone phase.

It is also possible to use grafted-block or block copolymers comprising at least one block of the polyorganosiloxane type and at least one block of a polyether. The polyorganosiloxane block may be a polydimethylsiloxane or a poly(C₂-C₁₈)alkylmethyl-siloxane; the polyether block may be a poly(C₂-C₁₈)alkylene, such as polyoxyethylene and/or polyoxypropylene. In one embodiment, dimethicone copolyols or (C₂-C₁₈)alkyldimethicone copolyols such as those sold under the name “Dow Corning 3225C” by the company Dow Corning, and lauryl methicones such as those sold under the name “Dow Corning Q2-5200” by the company Dow Corning, may also be used.

Grafted-block or block copolymers that may also be mentioned include those comprising at least one block resulting from the polymerization of at least one ethylenic monomer containing one or more optionally conjugated ethylenic bonds, for instance ethylene or dienes such as butadiene and isoprene, and at least one block of a vinyl polymer and a styrene polymer. When the ethylenic monomer comprises several optionally conjugated ethylenic bonds, the residual ethylenic unsaturations after the polymerization are generally hydrogenated. Thus, in a known manner, the polymerization of isoprene leads, after hydrogenation, to the formation of an ethylene-propylene block, and the polymerization of butadiene leads, after hydrogenation, to the formation of an ethylene-butylene block. Among these polymers that may be mentioned are block copolymers, including 1) those of the “diblock” or “triblock” type such as polystyrene/polyisoprene (SI), polystyrene/polybutadiene (SB) such as those sold under the name “Luvitol HSB” by BASF; 2) those such as polystyrene/copoly(ethylene-propylene) (SEP) such as those sold under the name “Kraton” by Shell Chemical Co.; or 3) those such as polystyrene/copoly(ethylene-butylene) (SEB). Examples of such polymers inclulde Kraton G1650 (SEBS), Kraton G1651 (SEBS), Kraton G1652 (SEBS), Kraton G1657X (SEBS), Kraton G1701X (SEP), Kraton G1702X (SEP), Kraton G1726X (SEB), Kraton D-1101 (SBS), Kraton D-1102 (SBS) and Kraton D-1107 (SIS). These polymers are generally known as hydrogenated or non-hydrogenated diene copolymers.

Gelled Permethyl 99A-750, 99A-753-59 and 99A-753-58 (mixture of triblock and of star polymer), Versagel 5960 from Penreco (triblock+star polymer); OS129880, OS129881 and OS84383 from Lubrizol (styrene/(meth)acrylate copolymer) may also be used.

Grafted-block or block copolymers comprising at least one block resulting from the polymerization of at least one ethylenic monomer containing one or more ethylenic bonds and of at least one block of an acrylic polymer may be used. Of these polymers mention may be made of poly(methyl (meth)acrylate)/polyisobutylene diblock or triblock copolymers or grafted copolymers containing a poly(methyl (meth)acrylate) skeleton and polyisobutylene grafts.

Grafted-block or block copolymers comprising at least one block resulting from the polymerization of at least one ethylenic monomer containing one or more ethylenic bonds and of at least one block of a polyether such as a C₂-C₁₈ polyalkylene (such as polyethylene and/or polyoxypropylene) may be used. Of these polymers mention may be made of polyoxyethylene/polybutadiene or polyoxyethylene/polyisobutylene diblock or triblock copolymers.

When a random polymer is used as the at least one stabilizer, it may be chosen such that it has a sufficient groups making it soluble in the intended organic synthesis medium.

Copolymers based on alkyl acrylates or (meth)acrylates derived from C₁-C₄ alcohols and on alkyl acrylates or (meth)acrylates derived from C₈-C₃₀ alcohols may thus be used. Mention may also be made of stearyl (meth)acrylate/methyl (meth)acrylate copolymer.

When the synthesis medium is apolar, the at least one stabilizer may be chosen so that the stabilizer provides the fullest possible coverage of the particles, several polymer-stabilizer chains then being absorbed onto a particle of polymer obtained by polymerization.

In this case, the at least stabilizer that is either a grafted polymer or a block polymer may be used so as to have better interfacial activity. Blocks or grafts that are insoluble in the synthesis solvent may provide bulkier coverage at the surface of the particles.

When the liquid synthesis solvent comprises at least one silicone oil, the at least one stabilizer may be chosen from one or more of grafted-block or block copolymers comprising at least one block of the polyorganosiloxane type and at least one block of a free-radical polymer or of a polyether or of a polyester, for instance polyoxypropylene and/or polyoxyethylene blocks.

When the liquid organic phase does not comprise any silicone oil, the at least one stabilizer may be chosen from one or more of:
(a) grafted-block or block copolymers comprising at least one block of polyorganosiloxane type and at least one block of a free-radical polymer or of a polyether or a polyester,
(b) copolymers of alkyl acrylates or (meth)acrylates derived from C₁-C₄ alcohols and of alkyl acrylates or (meth)acrylates derived from C₈-C₃₀ alcohols,
(c) grafted-block or block copolymers comprising at least one block resulting from the polymerization of at least one ethylenic monomer containing conjugated ethylenic bonds, and at least one block of a vinyl or acrylic polymer or of a polyether or of a polyester.

In one embodiment, diblock polymers are used as the at least one stabilizer.

At Least One Plasticizer

When the polymer has a glass transition temperature that is too high for the intended application, at least one plasticizer may be combined therewith. The at least one plasticizer may be chosen from plasticizers usually used in the field of application and from compounds liable to be solvents for the polymer. Coalescers may also be used in order to aid the polymer to form a continuous and homogeneous deposit.

The coalescers or plasticizers that may be used in the disclosure may include those mentioned in French Patent Publication FR 2 782 917.

The composition according to the disclosure may comprise at least one ester of at least one carboxylic acid containing from 1 to 7 carbon atoms and of a polyol comprising at least four hydroxyl groups, the ester having a molar mass of less than 5000 g/mol.

The polyol may be a monosaccharide or a polysaccharide comprising 1 to 10 saccharides, such as, for example, from 1 to 4. In one embodiment, the polyol may be a monosaccharide or a polysaccharide comprising one or two saccharides.

In another embodiment, the polyol according to the disclosure may be a disaccharide. Among the disaccharides that may be used, non-limitation mention may be made of sucrose (α-D-glucopyranosyl-(1-2)-β-D-fructofuranose), lactose (β-D-galactopyranosyl-(1-4)-β-D-glucopyranose) and maltose (α-D-glucopyranosyl-(1-4)-β-D-glucopyranose).

According to one embodiment, the ester is sucrose diacetate hexakis(2-methylpropanoate).

The ester may be liquid at room temperature and atmospheric pressure. The ester may be present in an amount ranging from 0.1% to 25% by weight, such as, for example, from 0.5% to 15% by weight, or from 3% to 15% by weight.

The mass ratio between the polymer particles and the ester of acid and polyol may range from 0.5 to 100, such as, for example, from 1 to 50, from 1 to 10, or from 1 to 5.

At Least One Fatty Phase

In one embodiment of the present disclosure, the at least one fatty phase of the composition may comprise at least one cosmetically or dermatologically acceptable and generally physiologically acceptable oil, chosen from, for example, carbon-based oils, hydrocarbon-based oils, fluoro oils and/or silicone oils of mineral, plant or synthetic origin.

As used herein, the term “oil” means any non-aqueous medium that is liquid at room temperature (25° C.) and atmospheric pressure (760 mmHg).

The total fatty phase of the composition may be present in an amount ranging from 5% to 90%, such as, for example, from 20% to 85%, of the total weight of the composition. In one embodiment, the total fatty phase of the composition may comprise at least 30% of the total weight of the composition.

According to one embodiment, the at least one fatty phase may be free of or contain less than 50% by weight of at least one volatile oil relative to the total weight of the fatty phase. In another embodiment, the at least one fatty phase contains less than 40%, such as, for example, less than 30%, less than 20%, or less than 10% by weight of at least one volatile oil relative to the total weight of the at least one fatty phase.

At Least One Apolar or Sparingly Polar Oil

According to one embodiment, the at least one fatty phase may comprise at least one apolar or sparingly polar oil, which is present in an amount comprising at least 5% by weight relative to the total weight of the composition. In particular, the oils of the at least one fatty phase may be non-volatile hydrocarbon-based apolar or sparingly polar oils, which are, for example, hydrocarbon-based.

In one embodiment, the apolar oils may have a solubility parameter δₐ=0.

The term “polar oil” means an oil composed of chemical compounds comprising at least one polar group. The “polar groups” are well known to those skilled in the art; they may be, for example, ionic polar groups or nonionic groups chosen from —COOH; —OH; ethylene oxide; propylene oxide; —PO₄; —NHR; —NR₁R₂ with R₁ and R₂ optionally forming a ring and representing a linear or branched C₁ to C₂₋₀ alkyl or alkoxy radical.

The sparingly polar oils may comprise oils that have a mean solubility parameter at 25° C. of: 0<δₐ<5.0 (J/cm³)^{1/2}.

The highly polar oils may have a mean solubility parameter δₐ according to the Hansen solubility space, at 25° C., of: δₐ≦5.0 (J/cm³)/^{1/2}.

The definition and calculation of the solubility parameters in the three-dimensional Hansen solubility space are described in the article by C. M. Hansen: “The three dimensional solubility parameters” J. Paint Technol. 39, 105 (1967).

According to this Hansen space:
δ_{D} characterizes the London dispersion forces derived from the formation of dipoles induced during molecular impacts;
δₚ characterizes the Debye interaction forces between permanent dipoles and the Keesom interaction forces between induced dipoles and permanent dipoles;
δₕ characterizes the specific interaction forces (such as hydrogen bonding, acid/base, donor/acceptor, etc.);
δₐ is determined by the equation: δₐ=(δₚ²+δₕ²)^{1/2}.

The parameters δₚ, δₕ, δ_{D} and δₐ are expressed in (J/cm³)^{1/2}.

When the oily phase is a mixture of different oils, the solubility parameters of the mixture are determined from those of the compounds taken separately, according to the following relationships:
δ_{Dmixt}=Σxi δ_{Di}; δₚₘᵢₓₜ=Σxi δₚᵢ and Γₕₘᵢₓₜ=Σxi δₕᵢ
δₐₘᵢₓₜ=(δ²ₚₘᵢₓₜ+δ²ₕₘᵢₓₜ)^{1/2}
in which xi represents the volume fraction of compound i in the mixture.

It is within the capability of a person skilled in the art to determine the amounts of each oil to obtain an oily phase that satisfies the desired criteria.

In one embodiment of the present disclosure, the at least one apolar or sparingly polar oil may be hydrocarbon-based. The term “hydrocarbon-based oil” means an oil formed essentially from, or even consisting of, carbon and hydrogen atoms, and optionally oxygen and nitrogen atoms, and containing no silicon or fluorine atoms. It may contain alcohol, ester, ether, carboxylic acid, amine and/or amide groups.

In one embodiment, the at least one apolar or sparingly polar oil may be non-volatile. The term “non-volatile oil” means any oil having a non-zero vapor pressure at room temperature and atmospheric pressure, of less than 0.02 mmHg and such as, for example, less than 10⁻³ mmHg.

The at least one apolar or sparingly polar oil may be present in an amount comprising at least 5% by weight of the total weight of the composition. In one embodiment, the at least one apolar or sparingly polar oil may be present in an amount ranging from 5% to 80% by weight relative to the total weight of the composition, such as, for example, from 10% to 60% by weight, or from 10% to 40% by weight relative to the total weight of the composition.

In one embodiment, the at least one apolar or sparingly polar oil is hydrocarbon-based may be present in an amount ranging from 10% to 40% by weight, such as, for example, from 15% to 30% by weight relative to the total weight of the composition.

In one embodiment, the at least one apolar or sparingly polar oil may be a non-volatile apolar hydrocarbon-based oil.

According to one embodiment, the at least one apolar hydrocarbon-based oil may be free of hetero atoms. The term “hetero atom” means an atom other than carbon or hydrogen.

According to one embodiment, the non-volatile hydrocarbon-based apolar oil may be chosen from linear or branched saturated alkanes.

The non-volatile apolar or sparingly polar hydrocarbon-based oil may be chosen from hydrocarbon-based oils with a molar mass of ranging from 300 and 900 g/mol, such as, for example, from 350 to 800 g/mol.

According to one embodiment, the at least one apolar oil is a non-volatile hydrocarbon-based oil that may be chosen from linear or branched hydrocarbons such as liquid paraffin, liquid petroleum jelly and liquid naphthalene, hydrogenated polyisobutene, isoeicosane, squalane and decene/butene copolymers, and mixtures thereof.

According to one embodiment, the at least one fatty phase comprises from 30% to 70% by weight of apolar non-volatile hydrocarbon-based oil relative to the weight of the at least one fatty phase, such as, for example, from 40% to 60% by weight relative to the weight of the at least one fatty phase.

As other examples of non-volatile apolar or sparingly polar oils, non-limiting mention may be made of:
hydrocarbon-based oils of animal origin, for instance squalene;
hydrocarbon-based plant oils such as liquid triglycerides of fatty acids of at least 10 carbon atoms;
synthetic esters and ethers, such as fatty acids, for instance the oils of formula R₁(CO)ₓOR₂ in which R₁ may be an acid residue containing from 2 to 29 carbon atoms with x being 0 or 1 and R₂ may be a hydrocarbon-based chain containing from 3 to 30 carbon atoms, for instance tributyl acetyl citrate, oleyl erucate, 2-octyldodecyl behenate, triisoarachidyl citrate, isocetyl or octyldodecanyl stearoylstearate, n-propyl acetate, tridecyl trimellitate, diisocetyl dodecanedioleate or stearate, arachidyl propionate, dibutyl phthalate, propylene carbonate or octyidodecyl pentanoate; polyol esters, for instance vitamin F, sorbitan isostearate, glyceryl triisostearate or diglyceryl triisostearate;
mixtures thereof.

The at least one fatty phase of the composition may also comprise a non-volatile silicone oil chosen from:
polydimethylsiloxanes (PDMS) optionally comprising a C₃-C₄₀ alkyl or C₃-C₄₀ alkoxy chain or a phenyl radical; the polydimethylsiloxanes comprising phenyl radicals may be chosen from phenyl trimethicones;
optionally fluorinated polyalkylmethylsiloxanes, for instance polymethyltrifluoropropyldimethylsiloxanes,
polyalkylmethylsiloxanes substituted with functional groups such as hydroxyl, thiol and/or amine groups;
polysiloxanes modified with fatty acids, fatty alcohols or polyoxyalkylenes.

In one embodiment, the at least one fatty phase of the composition may contain less than 10% of one or more silicone oils. In another embodiment, the at least one fatty phase contains less than 5%, such as, for example, less than 3%, or less than 1% of one or more silicone oils.

At Least One Non-Volatile Highly Polar Oil

The at least one fatty phase may contain, besides the apolar or sparingly polar oil as described above, a highly polar non-volatile oil chosen from fatty acid esters of 7 to 29 carbon atoms, for instance diisostearyl malate, isopropyl palmitate, diisopropyl adipate, caprylic/capric acid triglycerides, for instance those sold by the company Stearineries Dubois or those sold under the names Miglyol 810, 812 and 818 by the company Dynamit Nobel, Shea butter oil, isopropyl myristate, butyl stearate, hexyl laurate, diisopropyl adipate, isononyl isononate, 2-hexyldecyl laurate, 2-octyldecyl palmitate, 2-octyldodecyl myristate or lactate, 2-diethylhexyl succinate, 2-ethylhexyl palmitate, 2-octyldodecyl stearate or castor oil; lanolic acid, lauric acid or stearic acid esters; higher fatty alcohols (of 7 to 29 carbon atoms) such as stearyl alcohol, linoleyl alcohol, linolenyl alcohol, isostearyl alcohol, 2-octyldodecanol, decanol, dodecanol, octadecanol or oleyl alcohol; higher fatty acids (of 7 to 29 carbon atoms) such as myristic acid, palmitic acid, stearic acid, behenic acid, oleic acid, linoleic acid, linolenic acid or isostearic acid; mixtures thereof.

These non-volatile highly polar oils may represent from 0.1% to 10% and better still from 1% to 5% of the total weight of the composition.

At Least One Volatile Oil of the Fatty Phase

One or more volatile oils may be included in the fatty phase of the composition according to the disclosure, provided that it is present in an amount comprising less than 50% by weight of the fatty phase. These oils may be hydrocarbon-based oils or silicone oils optionally comprising alkyl or alkoxy groups that are pendent or at the end of a silicone chain.

The term “volatile oil” means any oil having a vapor pressure, at room temperature and atmospheric pressure, of greater than 0.02 mmHg.

As volatile silicone oils that may be used in the disclosure, non-limiting mention may be made of linear or cyclic silicones having a viscosity at room temperature of less than 8 cSt, such as, for example, those containing from 2 to 7 silicon atoms, these silicones optionally comprising alkyl or alkoxy groups of 1 to 10 carbon atoms. As volatile silicone oils that may be used in the disclosure, further non-limiting mention may be made of octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane and decamethyltetrasiloxane, and mixtures thereof.

As the at least one volatile oil that may be used in an embodiment of the disclosure, non-limiting mention may be made of C₈-C₁₆ isoalkane oils (also known as iso-paraffins), for instance isododecane, isodecane or isohexadecane and, for example, the oils sold under the trade names Isopar and Permethyl, and isododecane (Permethyl 99A).

In one embodiment of the present disclosure, the at least one fatty phase of the composition may be present in an amount comprising less than 10% of one or more volatile oils. In other embodiments, the at least one fatty phase may be present in an amount comprising less than 5%, less than 3%, or less than 1% of one or more volatile oils.

At Least One Oil of High Molar Mass

According to one embodiment, the at least one fatty phase may contain, besides at least one apolar or sparingly polar oil, at least one oil of high molar mass, for example, ranging from 650 and 10,000 g/mol.

In one embodiment of the present disclosure, the composition may comprise from 2% to 30%, such as, for example, from 5% to 25% or from 5% to 15%, of at least one oil with a molar mass ranging from 650 to 10,000 g/mol, such as, for example, from 750 to 7500 g/mol.

In one embodiment of the present disclosure, the oil with a molar mass ranging from 650 to 10,000 g/mol may be chosen from:
polybutylenes such as Indopol H-100 (molar mass or MM=965 g/mol), Indopol H-300 (MM=1340 g/mol) and Indopol H-1500 (MM=2160 g/mol), sold or manufactured by the company Amoco;
hydrogenated polyisobutylenes such as Panalane H-300 E sold or manufactured by the company Amoco (M=1340 g/mol), Viseal 20 000 sold or manufactured by the company Synteal (MM=6000 g/mol) and Rewopal PIB 1000 sold or manufactured by the company Witco (MM=1000 g/mol);
polydecenes and hydrogenated polydecenes such as: Puresyn 10 (MM=723 g/mol) and Puresyn 150 (MM=9200 g/mol), sold or manufactured by the company Mobil Chemicals;
vinylpyrrolidone copolymers such as: the vinylpyrrolidone/1-hexadecene copolymer, Antaron V-216 sold or manufactured by the company ISP (MM=7300 g/mol);
esters such as:
a) linear fatty acid esters with a total carbon number ranging from 35 to 70, for instance pentaerythrityl tetrapelargonate (MM=697.05 g/mol);
b) hydroxylated esters such as polyglyceryl-2 triisostearate (MM=965.58 g/mol),
c) aromatic esters such as tridecyl trimellitate (MM=757.19 g/mol);
d) esters of branched C₂₄-C₂₈ fatty alcohol or fatty acids, such as those described in patent application EP-A-0 955 039, such as, for example, triisoarachidyl citrate (MM=1033.76 g/mol), pentaerythrityl tetraisononanoate (MM=697.05 g/mol), glyceryl triisostearate (MM=891.51 g/mol), glyceryl tris(2-decyl)tetradecanoate (MM=1143.98 g/mol), pentaerythrityl tetraisostearate (MM=1202.02 g/mol), polyglyceryl-2 tetraisostearate (MM=1232.04 g/mol) or pentaerythrityl tetrakis(2-decyl)tetradecanoate (MM=1538.66 g/mol);
e) diol dimer esters and polyesters, such as esters of a diol dimer and of a fatty acid, and esters of a diol dimer and of a diacid.
   The esters of a diol dimer and of a monocarboxylic acid may be obtained from a monocarboxylic acid containing from 4 to 34 carbon atoms and especially from 10 to 32 carbon atoms, these acids being linear or branched, and saturated or unsaturated.
   As illustrative examples of monocarboxylic acids that are suitable for the disclosure, non-limiting mention may be made of fatty acids.
   In one embodiment, the esters of a diol dimer and of a dicarboxylic acid may be obtained from a diacid dimer derived from the dimerization of an unsaturated fatty acid, such as, for example, fatty acids of C₈ to C₃₄, fatty acids of C₁₂ to C₂₂, fatty acids of C₁₈ to C₂₀, and fatty acids of C₁₈.
   In another embodiment, it may be the diacid dimer from which the diol dimer to be esterified is also derived.
   The diol dimer esters may be obtained from a diol dimer produced by catalytic hydrogenation of a diacid dimer as described above, for example hydrogenated dilinoleic diacid.
   As examples of diol dimer esters that may be used according to the present disclosure, non-limiting mention may be made of the esters of dilinoleic diacids and of dilinoleyl diol dimers sold by the company Nippon Fine Chemical under the trade name Lusplan DD-DA5® and DD-DA7®.
silicone oils such as phenylsilicones, for instance Belsil PDM 1000 from the company Wacker (MM=9000 g/mol);
oils of plant origin such as sesame oil (820.6 g/mol); and
mixtures thereof.
   At Least One Wax, Pasty Compound

In an embodiment of the present disclosure, the composition may comprise at least one wax.

A “wax” is a lipophilic fatty compound that is solid at room temperature (25° C.), which undergoes a reversible solid/liquid change of state, having a melting point of greater than or equal to 30° C., and having an anisotropic crystal organization in the solid state. The size of the crystals is such that the crystals diffract and/or scatter light, giving the composition a cloudy, more or less opaque appearance. By bringing the wax to its melting point, it is possible to make it miscible with oils and to form a microscopically homogeneous mixture, but, on returning the temperature of the mixture to room temperature, recrystallization of the wax in the oils of the mixture is obtained.

As used herein, the melting point of the wax corresponds to the temperature of the most endothermic peak observed by thermal analysis (DSC) as described in standard ISO 11357-3; 1999.

The melting point of the wax may be measured using a differential scanning calorimeter (DSC), for example the calorimeter sold under the name MDSC 2920 by the company TA Instruments.

The measuring protocol is, for example, as follows:

A sample of 5 mg of wax placed in a crucible is subjected to a first temperature increase ranging from −20° C. to 100° C., at a heating rate of 10° C./minute, and is then cooled from 100° C. to −20° C. at a cooling rate of 10° C./minute and is finally subjected to a second temperature increase ranging from −20° C. to 100° C. at a heating rate of 5° C./minute. During the second temperature increase, the variation of the difference in power absorbed by the empty crucible and by the crucible containing the sample of wax is measured as a function of the temperature. The melting point of the compound is the value of the temperature corresponding to the top of the peak of the curve representing the variation of the difference in power absorbed as a function of the temperature.

The term “apolar wax” means a hydrocarbon-based or silicone apolar wax.

The at least one wax may be chosen from hydrocarbon-based waxes, fluoro waxes and/or silicone waxes and may be of plant, mineral, animal and/or synthetic origin. In one embodiment, the at least one wax may have a melting point of greater than 45° C.

As wax that may be used in the composition of the disclosure, non-limiting mention may be made of beeswax, carnauba wax or candelilla wax, paraffin, microcrystalline waxes, ceresin or ozokerite; synthetic waxes, for instance polyethylene waxes or Fischer-Tropsch waxes, and silicone waxes, for instance alkyl or alkoxy dimethicones containing from 16 to 45 carbon atoms.

In an embodiment of the present disclosure, the composition may contain an apolar wax such as a hydrocarbon-based or silicone apolar wax.

In one embodiment, the composition may contain an apolar hydrocarbon-based wax. The term “apolar hydrocarbon-based wax” means a wax containing at least 95% by weight of chemical compounds free of polar groups. The “polar groups” are well known to those skilled in the art; they may be, for example, ionic or nonionic polar groups chosen from —COOH; —OH; ethylene oxide; propylene oxide; —PO₄; —NHR; —NR₁R₂ with R₁ and R₂ optionally forming a ring and representing a linear or branched C₁ to C₂₀ alkyl or alkoxy radical.

According to one embodiment, the hydrocarbon-based wax may contain at least 95% by weight of compounds free of hetero atoms. The term “hetero atom” means an atom other than carbon or hydrogen.

According to one embodiment, the apolar hydrocarbon-based wax may contain at least 95% by weight of chemical compounds consisting of carbon and hydrogen. These chemical compounds may be chosen from linear or branched saturated alkanes.

According to one embodiment, the apolar wax may be chosen from linear hydrocarbon-based waxes.

Among linear hydrocarbon-based waxes that may be used in the composition of the present disclosure, non-limiting mention may be made of ethylene polymers and copolymers, linear paraffin waxes and Fischer-Tropsch waxes.

Further non-limiting examples of hydrocarbon-based waxes that may be mentioned include Fischer-Tropsch waxes, which are also known as polymethylene waxes. Such waxes correspond to the formula CₙH₂ₙ₊₂.

According to one embodiment of the disclosure, the at least one wax according to the disclosure may be a polymethylene wax, for example the wax Cirebelle 505® manufactured by the company Sasol, with a melting point equal to 40° C.

The apolar wax may be a wax of polyoxyalkylenated silicone type, such as, for example, a silicone comprising at least one oxyalkylene group of the type (—CₓH₂ₓO)ₐ in which x may range from 2 to 6 and a may be greater than or equal to 2.

In one embodiment of the present disclosure, the oxyalkylenated silicones may be chosen from the compounds of general formulae (I), (II), (III), and (IV): 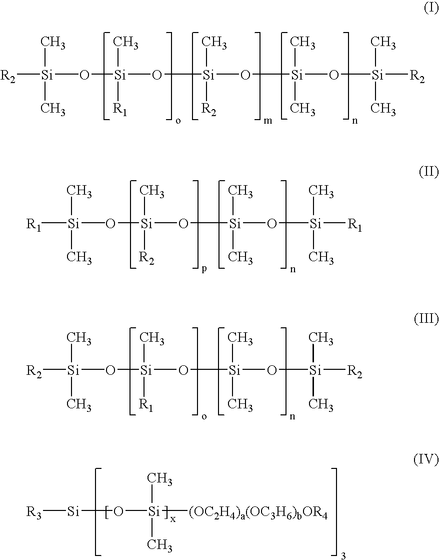
wherein, in formulae (I), (II), (III) and (IV):
R₁, which may be identical or different, is chosen from linear or branched C₁-C₃₀ alkyl and phenyl radicals;
R₂, which may be identical or different, is chosen from a radical C_{c}H_{2c}—O—(C₂H₄O)ₐ(C₃H₆O)_{b}—R₅ and a radical —C_{c}H_{2c}—O—(C₄H₈O)ₐ—R₅;
R₃ and R₄, which may be identical or different, may be chosen from a linear or branched C₁ to C₁₋₂ alkyl radical, such as, for example, a methyl radical;
R₅, which may be identical or different, may be chosen from a hydrogen atom, a linear or branched alkyl radical of 1 to 12 carbon atoms, a linear or branched alkoxy radical of 1 to 6 carbon atoms, a linear or branched acyl radical of 2 to 30 carbon atoms, a hydroxyl radical, a C₁-C₆ aminoalkoxy radical optionally substituted on the amine, a C₂-C₆ aminoacyl radical optionally substituted on the amine, an aminoalkyl radical optionally substituted on the amine and on the alkyl chain, a C₂-C₃₀ carboxyacyl radical, a group optionally substituted with one or two substituted aminoalkyl radicals, —NHCO(CH₂)_{d}OH, a phosphate group, -M, which may be identical or different, denotes a hydrogen atom, Na, K, Li, NH₄ and an organic amine;
d ranges from 1 to 10;
m ranges from 0 to 20;
n ranges from 0 to 500;
o ranges from 0 to 20;
p ranges from 1 to 50;
a ranges from 0 to 50;
b ranges from 0 to 50;
a+b is greater than or equal to 2;
c ranges from 0 to 4; and
x ranges from 1 to 100.

Non-limiting examples of such silicones are described, for example, in U.S. Pat. Nos. 5,070,171; 5,149,765; 5,093,452; and 5,091,493.

In one embodiment of the present disclosure, the silicones may be chosen from those of formula (III) in which R₂, which may be identical or different, is chosen from a radical C_{c}H_{2c}—O—(C₂H₄O)ₐ(C₃H₆O)_{b}—R₅, with R₅, a, b and c being defined as above. In this embodiment, b and c may be equal to 0 and a may range from 1 to 50, such as, for example, from 5 to 30, or from 10 to 20.

In an embodiment of the present disclosure, the low-melting apolar wax may be present in an amount ranging from 1% to 30%, such as, for example, from 3% to 20%, by weight relative to the total weight of the composition.

The mass ratio between the polymer particles and the low-melting apolar wax may range from 0.5 to 100, such as, for example, from 1 to 50, from 1 to 20, or from 3 to 15.

The composition according to the disclosure may also comprise at least one additional wax other than the low-melting apolar wax described above.

The at least one additional wax may have a melting point of greater than or equal to 65° C. It may be chosen from beeswax, carnauba wax, candelilla wax, paraffin, microcrystalline waxes, ceresin and ozokerite; polyethylene waxes, and Fischer-Tropsch waxes.

According to one embodiment, the apolar wax with a melting point of less than 65° C. (a) and the wax whose melting point is greater than or equal to 65° C. (b) may be present in a mass proportion (a)/(b) ranging from 30/70 to 55/45, such as, for example, from 40/60 to 45/55.

In an embodiment of the present disclosure, the amount of all the waxes contained in the composition may range from 15% to 35%, such as, for example, from 20% to 30%, by weight relative to the total weight of the composition.

The at least one additional wax may be an apolar wax with a melting point of greater than 65° C., for instance a microcrystalline wax, a polyethylene wax or a paraffin wax, or a mixture thereof.

The composition according to the disclosure may also contain a pasty compound. Synthesis medium for the polymer particles

According to one embodiment of the present disclosure, the at least one fatty phase of the composition may contain at least one oil, which may be the organic solvent serving as the polymerization medium for the polymer particles as described above.

The polymer dispersion may be manufactured as described in European Patent Publication EP 749 747.

In the described method, a mixture comprising the initial monomers and also a free-radical initiator is prepared. This mixture is dissolved in a solvent, which is referred to in the description hereinbelow as the “synthesis solvent”.

A synthesis solvent may be chosen such that the initial monomers and the free-radical initiator are soluble therein, and the polymer particles obtained are insoluble therein, such that they precipitate therefrom during their formation. In one embodiment, the synthesis solvent may be chosen from one or more alkanes such as heptane, isododecane or cyclohexane. The polymerization of the polymer particles may be performed in a synthesis solvent as described above, and the non-volatile hydrocarbon-based oil described above may then be added and the synthesis solvent may be selectively distilled off, provided that the non-volatile hydrocarbon-based oil is miscible with the synthesis solvent.

In one embodiment, the monomers may be present in the synthesis solvent, before polymerization, in a proportion of 5-20% by weight of the reaction mixture. The total amount of the monomers may be present in the solvent before the start of the reaction, or some of the monomers may be added gradually as the polymerization reaction proceeds.

In an embodiment of the present disclosure, the free-radical initiator may be azobisisobutyronitrile or tert-butylperoxy-2-ethyl hexanoate.

Hydrophilic Medium

The composition according to the disclosure may comprise a hydrophilic medium comprising water or a mixture of water and of at least one hydrophilic organic solvent, for instance alcohols and linear or branched lower monoalcohols containing from 2 to 5 carbon atoms, for instance ethanol, isopropanol or n-propanol, and polyols, for instance glycerol, diglycerol, propylene glycol, sorbitol, pentylene glycol and polyethylene glycols, or alternatively hydrophilic C₂ ethers and C₂-C₄ aldehydes.

The water or the mixture of water and of hydrophilic organic solvents may be present in the composition according to the disclosure in an amount ranging from 0.1% to 99% by weight, such as from 10% to 80% by weight, relative to the total weight of the composition.

Particulate Phase

The composition according to the disclosure may contain pigments and/or fillers.

The pigments may be white or colored, mineral and/or organic, and coated or uncoated. Among non-limiting examples of mineral pigments which may be mentioned are titanium dioxide, optionally surface-treated, zirconium oxide or cerium oxide, as well as zinc oxide, iron oxide (black, yellow or red), chromium oxide, manganese violet, ultramarine blue, chromium hydrate and ferric blue. Among non-limiting examples of organic pigments that may be mentioned are carbon black, pigments of the type such as organic lakes of barium, strontium, calcium or aluminium, including those submitted for certification by the Food and Drug Administration (FDA) (e.g. D&C or FD&C) and those exempt from FDA certification, for instance lakes based on cochineal carmine. The pigments may be present in an amount ranging from 0.1% to 50%, such as, for example, from 0.5% to 35%, or from 2% to 25%, of active material relative to the total weight of the composition.

In one embodiment, the nacreous pigments may be chosen from white nacreous pigments such as mica coated with titanium or with bismuth oxychloride, colored nacreous pigments such as titanium mica with iron oxides, titanium mica with, for example, ferric blue or chromium oxide, titanium mica with an organic pigment of the type mentioned above, as well as nacreous pigments based on bismuth oxychloride. They may be present in an amount ranging from 0 to 25% (of active material), for example, from 0.1% to 15% of the total weight of the composition (if present). Pigments with goniochromatic properties and/or pigments with a metallic effect as described in French Patent Application No. FR 0 209 246, the content of which is incorporated into the present patent application by reference, may thus be used.

The fillers may be mineral or organic, and lamellar or spherical. Non-limiting mention may be made of talc, mica, silica, kaolin, Nylon® powder (Orgasol® from Atochem), poly-α-alanine powder and polyethylene powder, powders of tetrafluoroethylene polymers (Teflon®), lauroyllysine, starch, boron nitride, hollow microspheres such as Expancel® (Nobel Industrie), Polytrap® (Dow Corning) and silicone resin microbeads (for example Tospearls® from Toshiba), precipitated calcium carbonate, magnesium carbonate, magnesium hydrocarbonate, hydroxyapatite, hollow silica microspheres (Silica Beads® from Maprecos), glass or ceramic microcapsules, metal soaps derived from carboxylic organic acids containing from 8 to 22 carbon atoms, such as, for example, from 12 to 18 carbon atoms, for example zinc stearate, magnesium stearate or lithium stearate, zinc laurate or magnesium myristate.

In an embodiment of the present disclosure, the composition may contain particles that are solid at room temperature, dispersed in the physiologically acceptable medium, introduced into the composition in the form of a colloidal dispersion, as described in Patent Application No. WO 02/39961, the content of which is incorporated by reference into the present patent application.

In another embodiment, the composition may contain at least one dispersant. The dispersant may serve to protect the dispersed filler or pigment particles against their agglomeration or flocculation. The concentration of dispersant generally used to stabilize a colloidal dispersion may range from 0.3 to 5 mg/m², such as, for example, from 0.5 to 4 mg/M², of surface area of pigment and/or filler particles. This dispersant may be a surfactant, an oligomer, a polymer or a mixture of several thereof, comprising one or more functionalities having strong affinity for the surface of the particles to be dispersed. In one embodiment, they can physically or chemically attach to the surface of the pigments. These dispersants may also have at least one functional group that is compatible with or soluble in the continuous medium. In another embodiment, esters of 12-hydroxystearic acid in particular and of a C₈ to C₂₀ fatty acid and of a polyol, for instance glycerol or diglycerol may be used, such as poly(12-hydroxystearic acid) stearate with a molecular weight of about 750 g/mol, such as the product sold under the name Solsperse 21,000 by the company Avecia, polyglyceryl-2 dipolyhydroxystearate (CTFA name) sold under the reference Dehymyls PGPH by the company Henkel, or polyhydroxystearic acid such as the product sold under the reference Arlacel P100 by the company Uniqema, and mixtures thereof.

As other dispersants that may be used in the composition of the disclosure, non-limiting mention may be made of quaternary ammonium derivatives of polycondensed fatty acids, for instance Solsperse 17,000 sold by the company Avecia, and mixtures of polydimethylsiloxane/oxypropylene such as those sold by the company Dow Corning under the references DC2-5185 and DC2-5225 C.

The polydihydroxystearic acid and the 12-hydroxystearic acid esters may be used for a hydrocarbon-based or fluorinated medium, whereas the mixtures of oxyethylene/oxypropylene dimethylsiloxane may be used for a silicone medium.

The colloidal dispersion may be a suspension of particles generally of micron size (<10 μm) in a continuous medium. The volume fraction of particles in a concentrated dispersion may range from 20% to 40%, such as, for example, greater than 30%, which corresponds to a weight content that may be up to 70% according to the density of the particles.

Additives and Galenical Forms

In an embodiment of the present disclosure, the composition may also contain one or more cosmetic or dermatological active agents such as those conventionally used.

As cosmetic, dermatological, hygiene or pharmaceutical active agents that may be used in the composition of the disclosure, non-limiting mention may be made of moisturizers, vitamins, essential fatty acids, sphingolipids and sunscreens. These active agents may be used in a usual amount for a person skilled in the art, such as, for example, from 0 to 20%, or from 0.001% to 15%, of the total weight of the composition.

The composition may also comprise any other additive usually used in such compositions, such as water, gelling agents, water-soluble dyes, antioxidants, fragrances, preserving agents and essential oils.

Needless to say, a person skilled in the art will take care to select this or these optional additional compounds, and/or the amount thereof, such that the beneficial properties of the composition according to the present disclosure are not, or are not substantially, adversely affected by the envisaged addition.

In one particular embodiment of the disclosure, the compositions according to the disclosure may be prepared in the usual manner by a person skilled in the art. They may be in the form of a cast product, for example in the form of a stick or tube, or in the form of a dish that may be used by direct contact or with a sponge. In an embodiment of the present disclosure, they may find an application as a cast foundation, a cast makeup rouge, a cast eyeshadow, a lipstick, base or balm to care for the lips, or a concealer product. They may also be in the form of a soft paste or alternatively a more or less fluid gel or cream, or a liquid, packaged in a tube. They may then constitute foundations, lipsticks, antisun products or skin-coloring products.

In one embodiment of the present disclosure, the composition of the disclosure may be anhydrous and, in this case, may contain less than 5% of water relative to the total weight of the composition.

In an embodiment of the present disclosure, these compositions for topical application may constitute a cosmetic, dermatological, hygiene or pharmaceutical composition for protecting, treating or caring for the face, the neck, the hands or the body (for example care cream, antisun oil or body gel), a makeup composition (for example a makeup gel, cream or stick) or a composition for artificially tanning or for protecting the skin.

The composition according to the disclosure may be in the form of a dermatological or care composition for the skin and/or the integuments or in the form of an antisun composition or a body hygiene composition, such as in deodorant form. When the composition is in such form, the composition may be uncolored. It may then be used as a care base for the skin, the integuments or the lips, such as lip balms, for protecting the lips against the cold and/or sunlight and/or the wind, or a care cream for the skin, the nails or the hair).

The compositions of the disclosure may be obtained by heating the various constituents to the melting point of the highest-melting waxes, followed by casting of the molten mixture in a mold (dish or finger stall). They may also be obtained by extrusion, as described in European Patent Application No. EP 0 667 146.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the embodiments disclosed herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosed embodiments are approximations, unless otherwise indicated the numerical values set forth in the specific examples are reported as precisely as possible. Any numerical value, however, inherently contain certain errors necessarily resulting from the standard deviation found in their respective testing measurements.

The embodiments disclosed herein are illustrated in greater detail by the examples described below.

### EXAMPLES 1 AND 2

### Lipstick

| | Example | Example | Comparative |
|---|---|---|---|
| Chemical name | 1 | 2 | formula |
| Polymer dispersion | 30 | 30 | 30 |
| 2-Decyltetradecanoic acid | 2.02 | 2.02 | 2.02 |
| triglyceride | | | |
| Dilinoleyl diol dimer/dilinoleyl | 10 | 10 | 10 |
| dimer copolymers | | | |
| Octyldodecanol | 9 | 9 | 9 |
| Preserving agents | 0.47 | 0.47 | 0.47 |
| Polycaprolactone of MW 1250 | 9 | — | — |
| g/mol (CAPA 1215 from Solvay) | | | |
| Polystearyl acrylate (Intelimer | — | 9 | — |
| IPA 13-1 from Landec) | | | |
| Polyvinyl laurate | — | — | 9 |
| Vinylpyrrolidone/eicosene | 6 | 6 | 6 |
| copolymer | | | |
| Microcrystalline wax | 10 | 10 | 10 |
| Polyethylene wax | 2 | 2 | 2 |
| Polymethylene wax of m.p. 40° C. | 10 | 10 | 10 |
| Pigments | 6.03 | 6.03 | 6.03 |
| Dimethicone-coated silica | 5 | 5 | 5 |
| Fragrance | 0.48 | 0.48 | 0.48 |
| TOTAL | 100 | 100 | 100 |

Synthesis of the Dispersion of Polymer Particles:

A dispersion of non-crosslinked copolymer of methyl acrylate and of acrylic acid in an 85/15 ratio, in heptane, was prepared according to the method of Example 1 of European Patent Publication No. EP 749 746. When the polymerization was complete, hydrogenated polyisobutene was added and the heptane was distilled off under vacuum.

A dispersion of poly(methyl acrylate/acrylic acid) particles stabilized in the hydrogenated polyisobutene with a polystyrene/copoly(ethylene-propylene) diblock copolymer sold under the name Kraton G1701, having a solids content of 21% by weight and a mean particle size equal to 150 nm, was thus obtained.

Procedure for Preparing the Lipstick

All the starting materials were weighed out in an oil-circulated jacketed heating pan and then heated with stirring (turbomixer).

After total melting of the materials and homogenization of the mixture, it was ground 5 times in succession on a three-roll mill. The paste obtained was left to stabilize for 24 hours at 20° C. and then packaged in heating bags.

Cosmetic Evaluation (In Vivo):

Formula 2 and the comparative formula were tested in half-lip tests on 7 women. The testers noted on a scale from 1 to 10 the level of migration of the formula after one hour (1=little, 10=substantial). The results are collated in the table below:

| | Example 2 | Comparative formula |
|---|---|---|
| | (polystearyl acrylate) | (polyvinyl laurate) |
| Migration | 1.14 | 3.43 |
| (average on | | |
| 7 individuals) | | |

After one hour of application, it was found that the formula containing the amorphous polymer migrated outside the contour of the lips.

The films of Examples 1 and 2 each containing a semi-crystalline polymer were also judged to be less tacky than that of the formula containing polyvinyl laurate.

Evaluation (In Vitro):

The three formulae were tested in vitro according to the test described above including evaluating the resistance of the formula to water and to oil. The results are as follows:

| | | | Comparative |
|---|---|---|---|
| | Example 1 | Example 2 | formula |
| | (polycapro- | (polystearyl | (polyvinyl |
| | lactone) | acrylate) | laurate) |
| Staying power under | 91.48 | 102.25 | 74.95 |
| pressure | | | |
| Staying power under | 53.96 | 60.43 | 40.69 |
| pressure + wiping | | | |

The formulae containing a semi-crystalline polymer and particles dispersed in a fatty phase predominantly containing a non-volatile oil had improved staying power in vitro (resistance to water and to oil) and also better cosmetic performance qualities (non-tacky and limited migration over time), when compared with those containing an amorphous polymer.

1. A cosmetic composition comprising:
at least one fatty phase;
a dispersion of particles of at least one polymer dispersed in the at least one fatty phase; and
at least one semi-crystalline polymer of organic structure having a melting point greater than or equal to 30° C.,
wherein the at least one fatty phase is free of volatile oil or contains less than 50% by weight of volatile oil relative to the weight of the at least one fatty phase.

2. The composition according to claim 1, wherein the at least one semi-crystalline polymer is chosen from homopolymers and copolymers comprising units resulting from the polymerization of at least one monomer bearing at least one crystallizable hydrophobic side chain.

3. The composition according to claim 1, wherein the at least one semi-crystalline polymer is chosen from homopolymers, copolymers, and a mixture of homopolymers and copolymers resulting from the polymerization of at least one monomer containing at least one crystallizable chain of formula X: 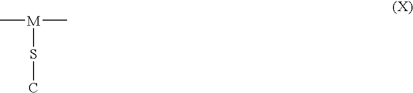
wherein:
M is an atom of the polymer skeleton;
S is a spacer; and
C is a crystallizable group;
wherein “S—C” represents an optionally fluorinated or perfluorinated alkyl chain comprising at least 11 carbon atoms.

4. The composition according to claim 1, wherein the at least one semi-crystalline polymer is chosen from polymers resulting from the polymerization of at least one monomer chosen from acrylic acid, (meth)acrylic acid, crotonic acid, itaconic acid, maleic acid, and maleic anhydride.

5. The composition according to claim 1, wherein the at least one semi-crystalline polymer is chosen from homopolymers and copolymers resulting from the polymerization of at least one monomer containing a crystallizable chain, chosen from C₁₄-C₂₄ saturated alkyl (meth)acrylates, C₁₁-C₁₅ perfluoroalkyl (meth)acrylates, C₁₄ to C₂₄ N-alkyl(meth)acrylamides optionally comprising a fluorine atom, vinyl esters comprising C₁₄ to C₂₄ alkyl chains, vinyl esters comprising C₁₄ to C₂₄ perfluoroalkyl chains, vinyl ethers comprising C₁₄ to C₂₄ alkyl chains, vinyl ethers comprising C₁₄ to C₂₄ perfluoroalkyl chains, C₁₄ to C₂₄ α-olefins, and para-alkylstyrenes having an alkyl group comprising from 12 to 24 carbon atoms.

6. The composition according to claim 1, wherein the at least one semi-crystalline polymer is chosen from alkyl(meth)acrylate and alkyl(meth)acrylamide homopolymers comprising a C₁₄ to C₂₄ alkyl group.

7. The composition according to claim 6, wherein the at least one semi-crystalline polymer is chosen from alkyl(meth)acrylate homopolymers comprising a C₁₆ to C₂₀ alkyl group.

8. The composition according to claim 2, wherein the at least one semi-crystalline polymer has a weight-average molar mass ranging from 5000 to 1,000,000.

9. The composition according to claim 8, wherein the at least one semi-crystalline polymer has a weight-average molar mass ranging from 10,000 to 800,000.

10. The composition according to claim 9, wherein the at least one semi-crystalline polymer has a weight-average molar mass ranging from 15,000 to 500,000.

11. The composition according to claim 1, wherein the at least one semi-crystalline polymer is a polyester polycondensate.

12. The composition according to claim 11, wherein the polyester polycondensate is chosen from polycaprolactones.

13. The composition according to claim 11, wherein the polyester polycondensate has a molar mass ranging from 200 to 10,000 g/mol.

14. The composition according to claim 13, wherein the polyester polycondensate has a molar mass ranging from 300 to 5000 g/mol.

15. The composition according to claim 14, wherein the polyester polycondensate has a molar mass ranging from 500 to 2000 g/mol.

16. The composition according to claim 8, wherein the at least one semi-crystalline polymer has a melting point ranging from 30° C. to 100° C.

17. The composition according to claim 16, wherein the at least one semi-crystalline polymer has a melting point ranging from 30° C. to 60° C.

18. The composition according to claim 1, wherein the at least one semi-crystalline polymer is present in an amount ranging from 0.1% to 80% of the total weight of the composition.

19. The composition according to claim 18, wherein the at least one semi-crystalline polymer is present in an amount ranging from 0.5% to 40% of the total weight of the composition.

20. The composition according to claim 19, wherein the at least one semi-crystalline polymer is present in an amount ranging from 3% to 30% of the total weight of the composition.

21. The composition according to claim 20, wherein the at least one semi-crystalline polymer is present in an amount ranging from 5% to 25% of the total weight of the composition.

22. The composition according to claim 1, wherein the polymer particles are solid and insoluble in the at least one fatty phase at a temperature of 25° C.

23. The composition according to claim 1, wherein the at least one polymer dispersed in the at least one fatty phase is not a wax.

24. The composition according to claim 1, wherein the particles of at least one polymer have a mean size ranging from 5 to 800 nm.

25. The composition according to claim 1, wherein the at least one polymer dispersed in the at least one fatty phase is film-forming.

26. The composition according to claim 1, wherein the at least one polymer dispersed in the at least one fatty phase is a hydrocarbon-based polymer.

27. The composition according to claim 1, wherein the particles of at least one polymer are insoluble in water-soluble alcohols.

28. The composition according to claim 1, wherein the particles of at least one polymer are chosen from polyurethanes, polyurethane-acrylics, polyureas, polyureas/polyurethanes, polyester-polyurethanes, polyether-polyurethanes, polyesters, polyester amides, fatty-chain polyesters, alkyds, acrylic polymers, acrylic copolymers, vinyl polymers, vinyl copolymers, acrylic-silicone copolymers, polyacrylamides, silicone polymers, fluoro polymers, and mixtures thereof.

29. The composition according to claim 1, wherein the at least one polymer dispersed in the at least one fatty phase is chosen from meth(acrylic)/(meth)acrylate copolymers.

30. The composition according to claim 29, wherein the at least one polymer dispersed in the fatty phase is chosen from acrylic/acrylate copolymers wherein the mass ratio of the acrylic units to acrylate units ranges from 0.1% to 40%.

31. The composition according to claim 30, wherein the at least one polymer dispersed in the at least one fatty phase is chosen from acrylic/acrylate copolymers wherein the mass ratio of the acrylic units to acrylate units ranges from 2% to 30%.

32. The composition according to claim 31, wherein the at least one polymer dispersed in the at least one fatty phase is chosen from acrylic/acrylate copolymers wherein the mass ratio of the acrylic units to acrylate units ranges from 5% to 20%.

33. The composition according to claim 1, wherein the at least one polymer dispersed in the at least one fatty phase is present, as solids, in an amount ranging from 5% to 40% of the total weight of the composition.

34. The composition according to claim 33, wherein the at least one polymer dispersed in the at least one fatty phase is present, as solids, in an amount ranging from 5% to 35% of the total weight of the composition.

35. The composition according to claim 34, wherein the at least one polymer dispersed in the at least one fatty phase is present, as solids, in an amount ranging from 8% to 30% of the total weight of the composition.

36. The composition according to claim 1, wherein the at least one fatty phase comprises less than 40% by weight of at least one volatile oil.

37. The composition according to claim 36, wherein the at least one fatty phase comprises less than 30% by weight of at least one volatile oil.

38. The composition according to claim 37, wherein the at least one fatty phase comprises less than 20% by weight of at least one volatile oil.

39. The composition according to claim 38, wherein the at least one fatty phase comprises less than 10% by weight of at least one volatile oil.

40. The composition according to claim 1, wherein the composition further comprises at least one stabilizer chosen from block polymers, grafted polymers and random polymers.

41. The composition according to claim 25, wherein the at least one stabilizer is chosen from a grafted-block and block polymer comprising at least one block resulting from the polymerization of diene and at least one block of a vinyl polymer.

42. The composition according to claim 41, wherein the at least one stabilizer is a diblock polymer.

43. The composition according to claim 1, wherein the composition further comprises at least one non-volatile apolar or sparingly polar oil.

44. The composition according to claim 43, wherein the at least one apolar or sparingly polar oil is present in an amount ranging from 5% to 80% by weight relative to the total weight of the composition.

45. The composition according to claim 44, wherein the at least one apolar or sparingly polar oil is present in an amount ranging from 10% to 60% by weight relative to the total weight of the composition.

46. The composition according to claim 45, wherein the at least one apolar or sparingly polar oil is present in an amount ranging from 10% to 40% by weight relative to the total weight of the composition.

47. The composition according to claim 46, wherein the at least one apolar or sparingly polar oil is present in an amount ranging from 15% to 30% by weight relative to the total weight of the composition.

48. The composition according to claim 43, wherein the at least one apolar or sparingly polar oil is a hydrocarbon-based apolar oil.

49. The composition according to claim 48, wherein the hydrocarbon-based apolar oil is chosen from oils with a molar mass ranging from 300 to 900 g/mol.

50. The composition according to claim 49, wherein the hydrocarbon-based apolar oil is chosen from oils with a molar mass ranging from 350 to 800 g/mol.

51. The composition according to claim 48, wherein the hydrocarbon-based apolar oil is chosen from one or more of linear and branched hydrocarbons, hydrogenated polyisobutene, isoeicosane, and squalane and decene/butene copolymers.

52. The composition according to claim 51, wherein the hydrocarbon-based apolar oil is chosen from one or more of liquid paraffin, liquid petroleum jelly and liquid naphthalene.

53. The composition according to claim 1, wherein the at least one polymer is dispersed in the at least one fatty phase in an amount sufficient to result in a mean gloss at 200 of a deposit of the composition spread onto a support, greater than or equal to 30 out of 100.

54. The composition according to claim 1, wherein the at least one polymer is dispersed in the at least one fatty phase is present in an amount to allow the composition to be capable of forming a deposit having a wear index of greater than or equal to 30%.

55. The composition according to claim 1, where the composition further comprises at least one pulverulent dyestuff chosen from pigments, nacres, and flakes.

56. The composition according to claim 1, wherein the composition is in the form of a product for caring for and/or making up the skin and/or the lips.

57. The composition according to claim 1, wherein the composition is in the form of a foundation, a makeup rouge, an eyeshadow, a lipstick, a care base or care balm for the lips, a concealer product, an eyeliner or a mascara.

58. A method for manufacturing a glossy, non-migrating makeup comprising:
dispersing particles of at least one polymer in at least one fatty phase;
mixing the dispersion of particles of at least one particle in at least one fatty phase with at least one semi-crystalline polymer of organic structure having a melting point greater than or equal to 30° C.,
wherein the at least one fatty phase is free of volatile oil or contains less than 50% by weight of volatile oil relative to the weight of the at least one fatty phase.
